(19) United States
(12) Patent Application Publication
(10) Pub. No.: US 2009/0126064 A1
(43) Pub. Date: May 21, 2009

(54) Mask and Face Shield

(76) Inventor: Brian K. Reaux, Red Oak, TX (US)

Correspondence Address: Brian K. Reaux, 710 Rock Hill Dr, Red Oak, TX, 75154, US

(21) Appl. No.: 12/252,109

(22) Filed: Oct. 15, 2008

Related U.S. Application Data
(60) Provisional application No. 60/999,313, filed on Oct. 17, 2007.
(60) Provisional application No. 61/002,321, filed on Nov. 8, 2007.
(60) Provisional application No. 61/137,667, filed on Aug. 1, 2008.

(51) Int. Cl. A61F 9/00; A41D 13/00

(52) U.S. Cl. 2/15; 2/9

(57) ABSTRACT

A mask and shield assembly for protecting the face of a user may include A reversible mask having an attachment device and a frame member for detachably connecting to the attachment device of the reversible mask. The frame member may include an outwardly extending projection, and the reversible mask includes an aperture, and the outwardly extending projection may cooperate with the aperture to hold the reversible mask in position. The reversible mask may include a first surface of the reversible mask including camouflage indicia, and the reversible mask includes a second surface including hunter orange indicia. The attachment device may include an expandable aperture and the expandable aperture may include a slit. The outwardly extending projection may include an enlarged end, and the frame member may include a concave curved portion.

### PRIORITY

The present invention claims priority under 35 USC section 119 and is based on the following provisional applications: 60/999,313 which was filed on Oct. 17, 2007, 61/002,321 which was filed on Nov. 8, 2007, 61/137,667 which was filed on Aug. 1, 2008 and 61/118,923 which was filed on Aug. 14, 2008.

### FIELD OF THE INVENTION

The present invention relates to mask and eye shields for caregivers, and more particularly to a reversible mask.

### BACKGROUND OF THE INVENTION

Transparent eye shields are especially useful in medical or dental surgical operations where the user's eyes may be exposed to blood-born pathogens such as Hepatitis, Staphylococcus and HIV viruses. Although such devices are available, they are constructed with the frame substantially permanently attached to or otherwise integral with the eye covering portion, which is typically of industrial grade plastic, suitable for protecting the eyes from flying debris, but unnecessarily thick and heavy for use in a medical or dental setting.

### SUMMARY

A mask and shield assembly for protecting the face of a user may include A reversible mask having an attachment device and a frame member for detachably connecting to the attachment device of the reversible mask. The frame member may include an outwardly extending projection, and the reversible mask includes an aperture, and the outwardly extending projection may cooperate with the aperture to hold the reversible mask in position.

The reversible mask may include a first surface of the reversible mask including camouflage indicia, and the reversible mask includes a second surface including hunter orange indicia.

The attachment device may include an expandable aperture and the expandable aperture may include a slit.

The outwardly extending projection may include an enlarged end, and the frame member may include a concave curved portion.

The frame member may be flexible, and the attachment device may include an adhesive surface.

The attachment surface may be protected by a protective sheet, and the adhesive surface may be positioned on a front and back of the adhesive device.

The mask may include an expandable edge member, and the expandable edge member may be positioned on a lower edge of the mask.

The expandable edge member may be positioned along a side edge of the mask, and the reversible mask may be affixed to the eye shielding member.

The shielding eye member may include a foldable side member, and the shielding eye member may include a protective cover.

The protective cover may include a tab member, and the reversible mask may include a lower cord member.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a top view of the frame member of the present invention;

FIG. 2 illustrates a top view of the frame member of the present invention;

FIG. 3 illustrates a cross-sectional view of the outwardly extending projection of the present invention;

FIG. 4 illustrates a perspective view of a reversible mask in accordance with the teachings of the present invention;

FIG. 5 illustrates a perspective view of another reversible mask in accordance with the teachings of the present invention;

FIG. 6 illustrates a user wearing the reversible mask;

FIG. 7 illustrates a detachable mask of the present invention;

FIG. 8 illustrates another detachable mask of the present invention;

FIG. 9 illustrates another detachable mask of the present invention;

FIG. 10 illustrates the expandable aperture of the present invention;

FIG. 13 illustrates an earpiece of the present invention;

FIG. 14 illustrates a perspective view of the detachable mask of the present invention;

FIG. 15 illustrates a cross-sectional view of the detachable mask and eye shield of the present invention;

FIG. 16 illustrates a perspective view of the detachable mask and eye shield of the present invention;

FIG. 17 illustrates a perspective view of the user of the detachable mask with a lower cord member;

FIG. 18 illustrates a perspective view of the user of the detachable mask without the lower cord member;

FIG. 19 illustrates a perspective view of the user of the detachable mask with the lower cord member;

FIG. 20 illustrates a perspective view of the user of the detachable mask without the lower cord member;

FIG. 21 illustrates a perspective view of the user of the detachable mask and the eye shield member with a protective cover;

FIG. 22 illustrates a side view of the eye shield member and protective cover;

FIG. 23 illustrates a top view of the eye shield member and protective cover;

FIG. 24 illustrates a perspective view of another mask of the present invention;

FIG. 25 illustrates a perspective view of a mask and eye shield member of the present invention;

FIG. 26 illustrates a cross-sectional view of the mask of the present invention;

FIG. 27 illustrates a cross-sectional view of the mask and eye shield of the present invention;

FIG. 28 illustrates a perspective view of the mask and eye shield of the present invention;

FIG. 29 illustrates a perspective view of another mask and eye shield of the present invention;

FIG. 30 illustrates a perspective view of the mask of the present invention;

FIG. 31 illustrates another perspective view of the mask and eyeglasses of the present invention;

FIG. 32 illustrates a top view of another frame member;

FIG. 33 illustrates a side view of the frame member;

FIG. 34 illustrates a top view of another frame member;

FIG. 35 illustrates a perspective view of another frame member;

FIG. 36 illustrates a side view of the frame member;

FIG. 37 illustrates a side view of the inward or outward projection member;

FIG. 38 illustrates a side view of another mask of the present invention;

FIG. 39 illustrates a perspective view of another mask of the present invention;

FIG. 40 illustrates a perspective view of another mask of the present invention;

FIG. 41 illustrates a frame member with the projections on the inside;

FIG. 42 illustrates a frame member with the projections on the outside;

FIG. 43 illustrates a protective cover with camouflage design;

FIG. 44 illustrates a protective cover that is clear.

### DETAILED DESCRIPTION

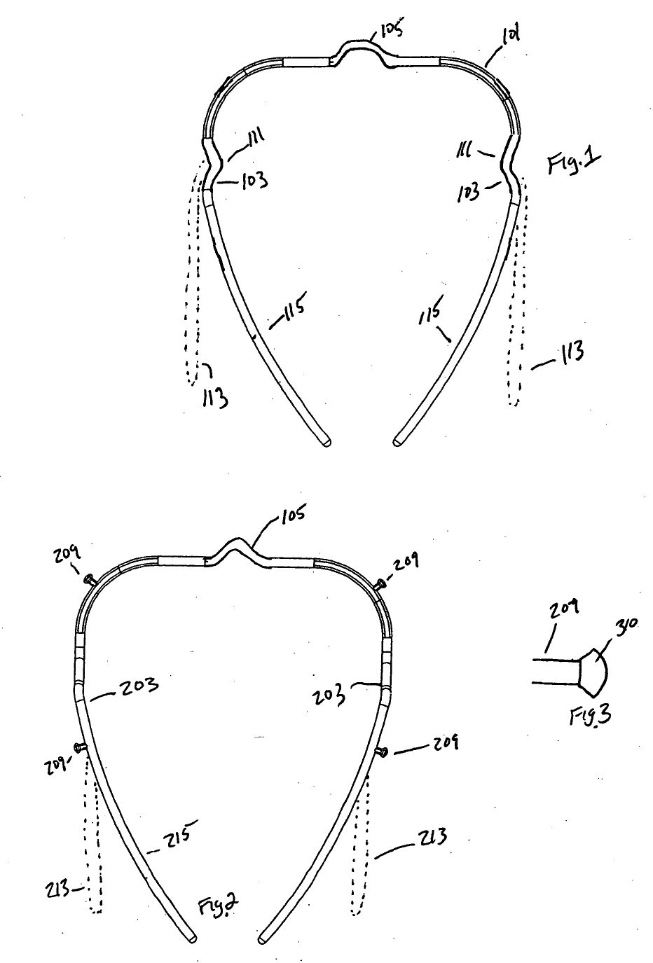

FIG. 1 illustrates a frame member 101 which is approximately C or U shaped. The frame member 101 may include a front portion 107 which may cooperate with the forehead and nose of the user. The front portion 107 may include a concaved curve portion 105, and the frame member 101 may include a temple portion 103 to cooperate with the side of the head of the user and may be positioned approximately at the temple of the user. The temple portion 103 may include an approximately S-shaped portion 111 to provide flexibility to the temple portion 103. FIG. 1 illustrates that the temple portion 103 may be flexible to move between a first position 113 and a second position 115 which may be closer to the head of the user.

FIG. 2 illustrates a frame member 201 which is approximately C shaped. The frame member 201 may include a front portion 207 which may cooperate with the forehead and nose of the user. The front portion 207 may include a concaved curve portion 205, and the frame member 201 may include a temple portion 203 to cooperate with the side of the head of the user and may be positioned Approximately at the temple of the user. FIG. 2 illustrates that the temple portion 203 may be flexible to move between a first position 213 and a second position 215 which may be closer to the head of the user.

FIG. 2 illustrates that the temple portion 203 and the front portion 207 may include an outwardly extending projection 209 which may extend substantially outwards or transverse to the surface of the temple portion 203 or the front portion 207.

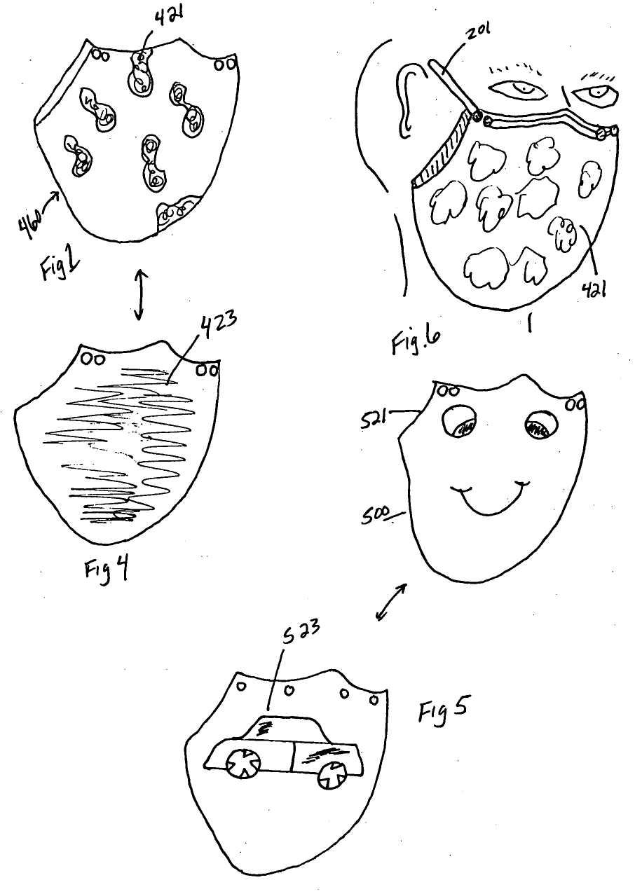

FIG. 3 illustrates the outwardly extending projection 209 and illustrates that the outwardly extending projection 209 may include an enlarged radius end member 3102 allowing the mask (not shown) to be held in place.

FIG. 4 illustrates a reversible mask 400 which may include a first surface 421 which may include indicia of camouflage design which may correspond to the first side of the reversible mask 400 and which may include a second surface 423 which may include indicia of hunter orange design which may correspond to the second side of the reversible mask 400.

FIG. 5 illustrates a reversible mask 500 which may include a first surface 521 which may include indicia of a happy face design which may correspond to the first side of the reversible mask 500 and which may include a second surface 523 which may include indicia of an automobile design which may correspond to the second side of the reversible mask 500.

FIG. 6 illustrates the reversible mask 400 affixed to the frame member 201 with the first surface 421 which may correspond to the camouflage design.

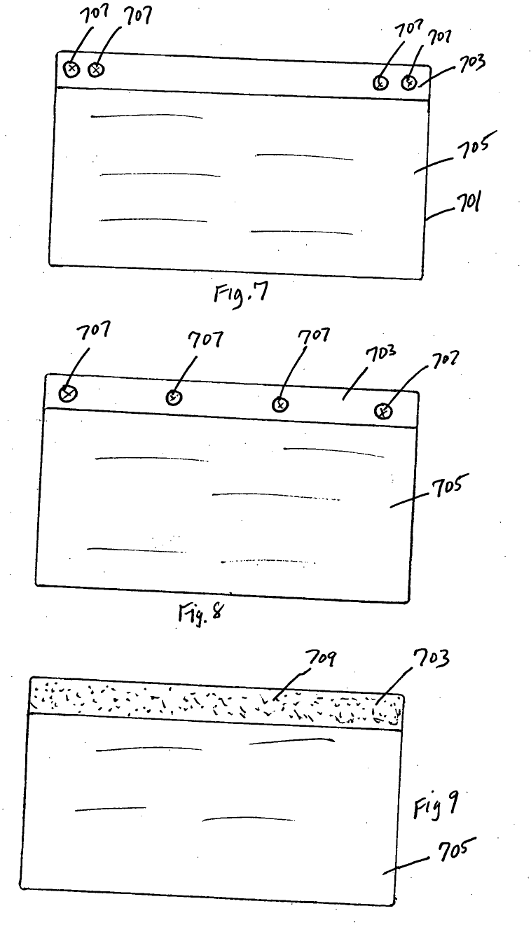

FIG. 7 illustrates a detachable mask 701 and illustrates the front surface 705 of the detachable mask 701. The detachable mask 701 may include an attachment device 703 which may be attached the detachable mask 701 to a detachable shield (not shown). The attachment device 703 may include expandable apertures 707 which may be positioned together to attach to the frame member 101. The detachable mask 701 may be adapted for frame member with no projecting members.

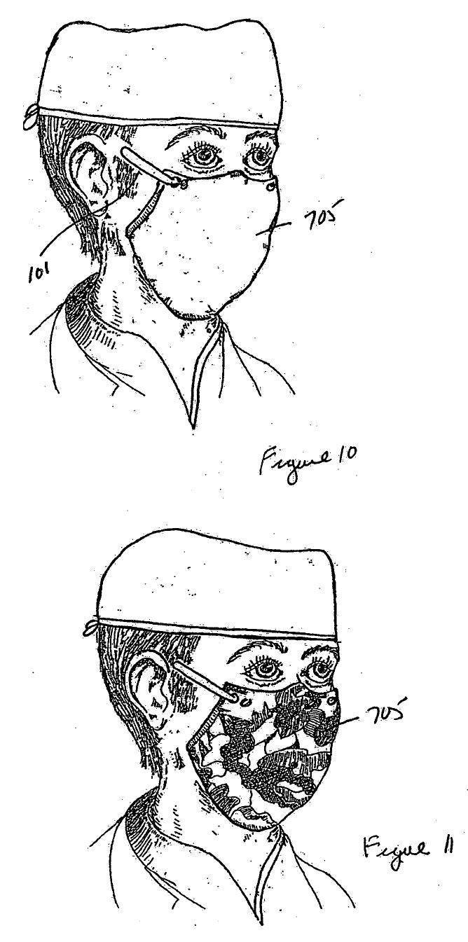

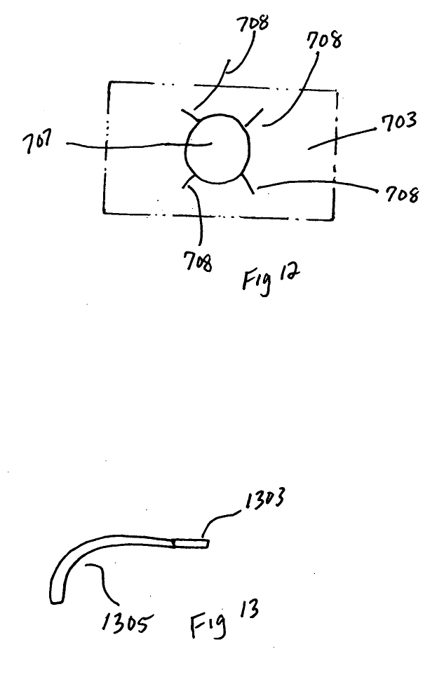

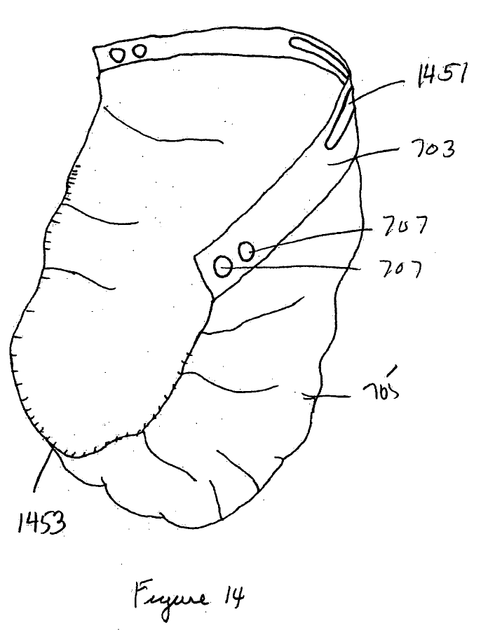

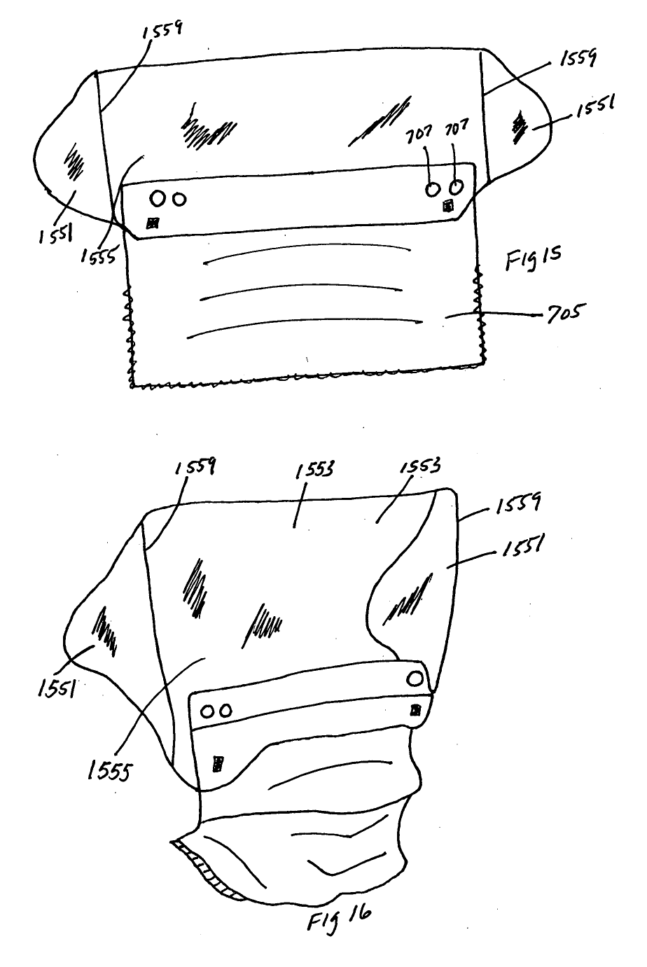

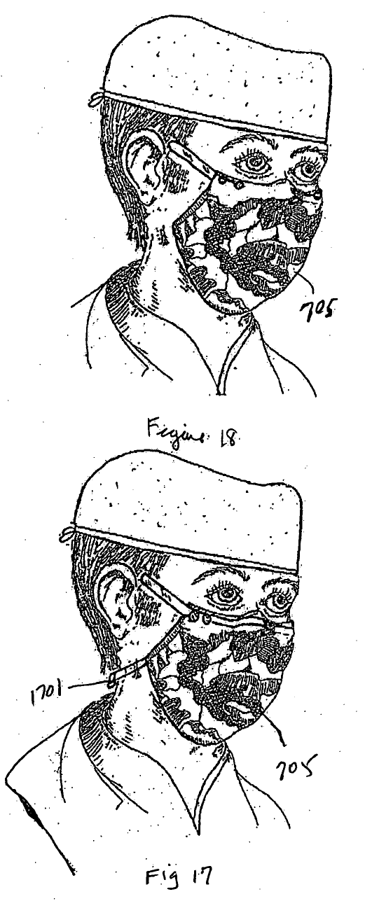

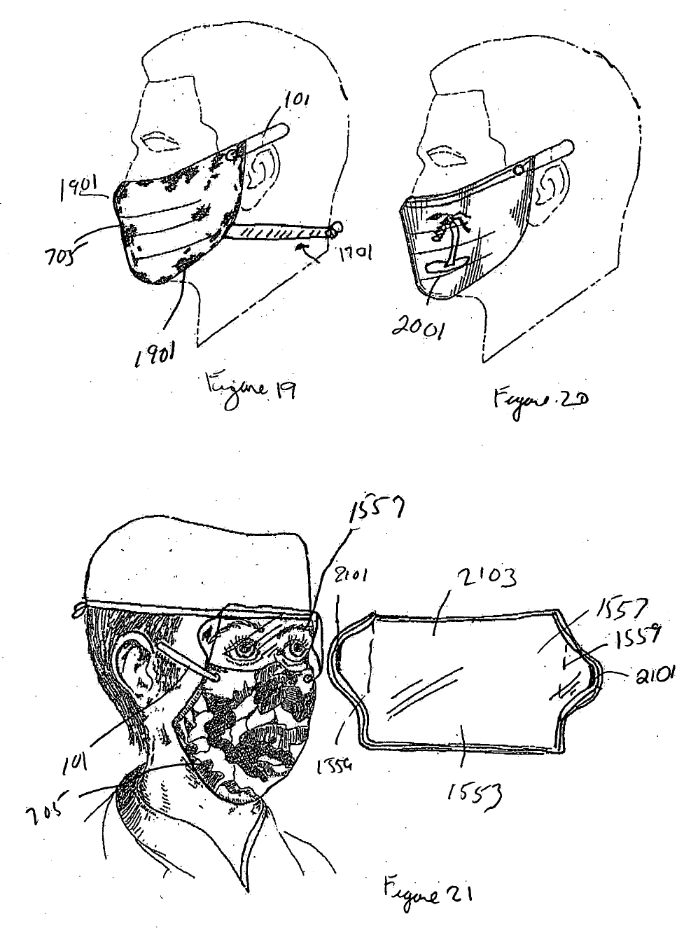

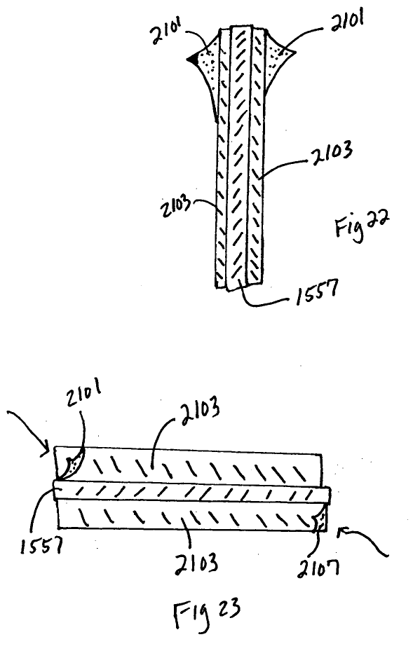

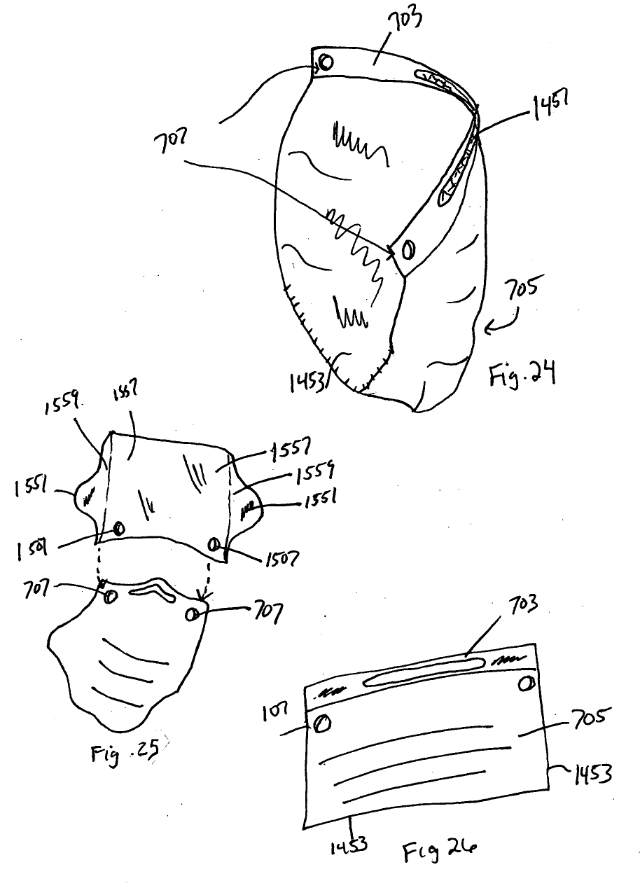

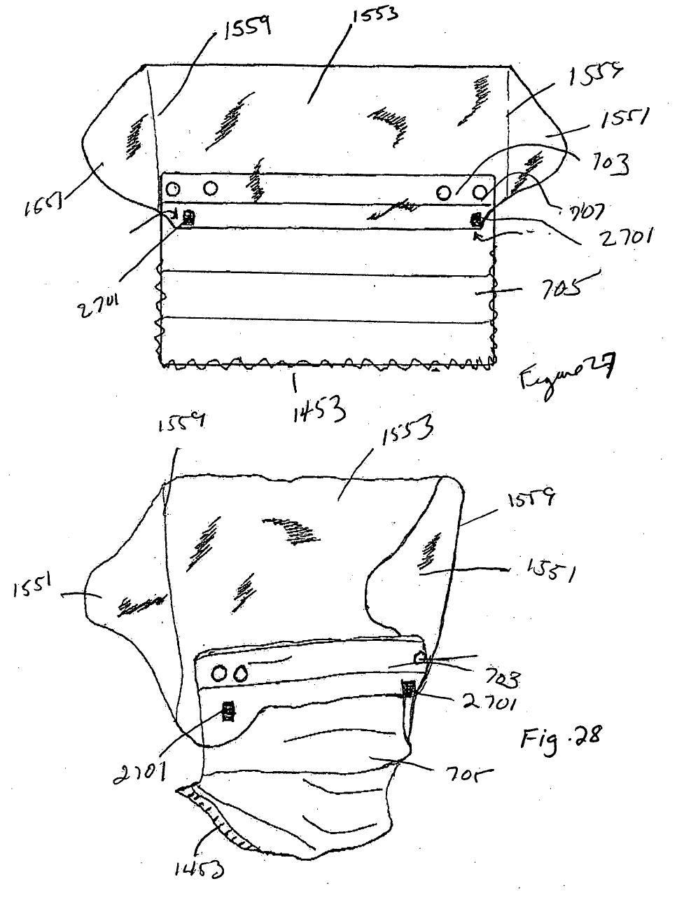

FIG. 8 illustrates another detachable mask 705 which may include an attachment device 703 which may include expandable apertures 707 which may be substantially equally spaced for attachment to the frame member 101. The detachable mask may be adapted for a frame member with projecting members (outwards or inwards)

FIG. 9 illustrates another detachable mask 705 which may include the attachment device 703 which may include an adhesive surface 709 which may be on one side of the detachable mask 705 or maybe on both sides of the detachable mask 705. The adhesive surface 709 may be covered with a detachable sheet to protect the adhesive surface 709 until it is ready to be used.

FIG. 10 illustrates the user wearing the detachable mask 705 which is detachably connected to the frame member 701. The detachable mask 705 may be reversible and is illustrated having a hunters orange surface.

FIG. 11 illustrates the user wearing a detachable mast 705 which is the detachably connected to the frame member 701, and the detachable mask 705 may include a reversible surface. FIG. 11 shows the detachable mask 705 having camouflage indicia. The camouflage indicia may be appropriate under battlefield conditions when it may be important to remain hidden.

FIG. 12 illustrates the expandable aperture 707 which may include slits 708 which allow the expandable aperture 707 to expand.

FIG. 13 illustrates a portion of the earpiece member 1301 of the present invention. The earpiece member 1301 may include a narrow portion 1303 and a thicker portion 1305 which may be connected to the narrow portion 1303. The earpiece member 1301 may be an end to the temple portion 103.

FIG. 14 illustrates the detachable mask 705 which may be reversible. FIG. 14 additionally illustrates that the detachable mask 705 may include the attachment device 703 which may include expandable aperture 707 to cooperate with the outwardly extending projection 709.

FIG. 14 additionally illustrates that the detachable mask 705 may include an expandable edge member 1453 which may be elastic or other suitable material in order to retract and expand the edge of the detachable mask 705 to the face of the user. More particularly, while the expandable edge member 1453 may be used on any edge of the detachable mask 705, FIG. 14 illustrates that the expandable edge member 1453 may be used on the lower and side edges of the detachable mask 705 in order to provide a relatively snug fit with the face of the user.

FIG. 14 additionally illustrates that the attachment device 703 may include a reinforcing member 1451 which may be a band formed from metal, wood, plastic or other suitable material in order to reinforce and conform the detachable mask 705 to the face of the user. The reinforcing member 1451 may extend along a portion of the attachment device 703 or may extend the entire length of the attachment device 703.

FIG. 15 illustrates an eye shield member 1557 which may be detachably connected to the detachable mask 705 and which may include eye shield apertures 1507 which may cooperate and align with the expandable aperture 707. The eye shield member 1557 may include a front member 1553 which may be a substantially planar member which may shield the eyes and forehead of the user. The front member 1553 may be defined along the sides of the front member 1553 by a fold line 1559 which may be a weakened portion of the eye shield member 1557 to allow the foldable side member 1512 to fold or rotate so that the foldable side member 1512 may cooperate and cover a portion of the side of the face and to additionally enclose the eye shield member 1557 around the face of the user.

FIG. 16 illustrates a perspective view of the eye shield member 1557 which may be detachably connected to the detachable mask 705 and which may include eye shield apertures 1507 which may cooperate and align with the expandable aperture 707. The eye shield member 1557 may include a front member 1553 which may be a substantially planar member which may shield the eyes and forehead of the user. The front member 1553 may be defined along the sides of the front member 1553 by a fold line 1559 which may be a weakened portion of the eye shield member 1557 to allow the foldable side member 1512 to fold or rotate so that the foldable side member 1512 may cooperate and cover a portion of the side of the face and to additionally enclose the eye shield member 1557 around the face of the user.

FIG. 16 illustrates that the foldable side members 1551 has been rotated approximately 90° in order to further protect the face of the user.

FIG. 17 illustrates the detachable mask 705 which may include a lower cord member 1701 which may be flexible and/or expandable and may be formed from elastic or may be flexible and may not be expandable in order to extend around the head of the user. The detachable mask 705 may include the lower cord member 1701 on opposing sides of the detachable mask 705 so that the lower cord member 1701 may be tied to maintain the detachable mask 705 on the face of the user.

FIG. 18 illustrate of the detachable mask 705 without the lower cord member 1701.

FIG. 19 illustrates the detachable and reversible mask 705 which may include color indicia 1901 to provide a camouflage design.

FIG. 20 illustrates the detachable and reversible mask 705 which may be reversed to include color indicia 2001 to provide a color scene design. FIG. 20 illustrates the detachable and reversible mask 705 without the lower cord member.

FIG. 21 illustrates that the detachable mask 705 may be detachably connected to the frame member 101 and the eye shield member 1557. FIG. 21 additionally illustrates the eye shield member 1557 which may include a front member 1553 which may be defined by fold lines 1559 to allow the foldable side member 1551 to fold or rotate from a position substantially planar with the front member 1553 to a position substantially 90° to the front member 1553. FIG. 21 additionally illustrates that the eye shield member 1557 may include a protective cover 2103 which is detachably connected to the eye shield 1557 by a removable adhesive or other appropriate device and may be removed by pulling on the tab member 2101 of the protective cover 2103. The tab member 2101 may extend beyond the eye shield member 1557.

FIG. 22 illustrates a side view of the eye shield member 1557, the protective cover 2103 and the tab member 2101.

FIG. 22 additionally illustrates that the eye shield member 1557 may include a protective cover 2103 which is detachably connected to the eye shield 1557 by a removable adhesive or other appropriate device and may be removed by pulling on the tab member 2101 of the protective cover 2103, and the tab member 2101 may extend beyond the eye shield member 1557. The eye shield member 1557 may be formed from transparent material and may be flexible or may be rigid.

FIG. 23 illustrates a top view of the eye shield member 1557, the protective cover 2103 and the tab member 2101.

FIG. 23 additionally illustrates that the eye shield member 1557 may include a protective cover 2103 which is detachably connected to the eye shield 1557 by a removable adhesive or other appropriate device and may be removed by pulling on the tab member 2101 of the protective cover 2103, and the tab member 2101 may extend beyond the eye shield member 1557.

FIG. 24 illustrates a mask 705 which may include an attachment device 703, a reinforcing member 1451 and a single expandable aperture 707 at each end of the attachment device 703.

FIG. 25 illustrates a mask 705 which may include an attachment device 703, a reinforcing member 1451 and a single expandable aperture 707 at each end of the attachment device 703. FIG. 25 additionally illustrates an eye shield member 1557 which cooperates with the mask 705 and may include a single by shield aperture 1507 which may cooperate with the single expandable aperture 707. FIG. 25 additionally illustrates the foldable side member 1551, the front member 1553 and the fold line 1559.

FIG. 26 illustrates a mask 705 which may include an attachment device 703, a reinforcing member 1451 and a single expandable aperture 707 at each end of the attachment device 703.

FIG. 27 illustrates a mask 705 which may include an attachment device 703, a reinforcing member 1451 and an expandable aperture 707 at each end of the attachment device 703 and across the surface of the attachment device 703. FIG. 27 additionally illustrates an eye shield member 1557 which cooperates with the mask 705. FIG. 27 additionally illustrates the foldable side member 1551, the front member 1553 and the fold line 1559. FIG. 27 illustrates that the detachable and reversible mask 705 may be affixed to the eye shield member 1557 by welding, adhesive or other materials.

FIG. 28 illustrates a mask 705 which may include an attachment device 703, a reinforcing member 1451 and an expandable aperture 707 at each end of the attachment device 703 and across the surface of the attachment device 703. FIG. 28 additionally illustrates an eye shield member 1557 which cooperates with the mask 705. FIG. 28 additionally illustrates the foldable side member 1551, the front member 1553 and the fold line 1559. FIG. 28 illustrates that the detachable and reversible mask 705 may be affixed to the eye shield member 1557 by welding, adhesive or other materials.

FIG. 29 illustrates a perspective view of a mask 2905 which cooperates with the eye shield member 1557 and which may be detachably connected or affixed to the eye shield member 1577. FIG. 29 illustrates that the mask 2905 may include the upward extending mask portion 2901. The outward extending mask portion 2901 may include a first and second expandable aperture 707 in order to cooperate with ear pieces (shown as eyeglasses 2915 in FIG. 31) and maintain the mask 2905 in position.

FIG. 29 illustrates that the mask 2905 may include a lower cord member 1701.

FIG. 30 illustrates a perspective view of a mask 2905 which cooperates with the eye shield member 1557 and which may be detachably connected or affixed to the eye shield member 1577. FIG. 30 illustrates that the mask 2905 may include the upward extending mask portion 2901. The outward extending mask portion 2901 may include a first and second expandable aperture 707 in order to cooperate with ear pieces (shown as eyeglasses 2915 in FIG. 31) and maintain the mask 2905 in position.

FIG. 30 illustrates that the mask 2905 may not include a lower cord member 1701.

FIG. 31 illustrates a perspective view of a mask 2905 which cooperates with the eye shield member 1557 and which may be detachably connected or affixed to the eye shield member 1577. FIG. 29 illustrates that the mask 2905 may include the upward extending mask portion 2901. The outward extending mask portion 2901 may include a first and second expandable aperture 707 in order to cooperate with ear pieces of the eyeglasses 2915 and maintain the mask 2905 in position.

FIG. 31 illustrates that the mask 2905 may include a lower cord member 1701.

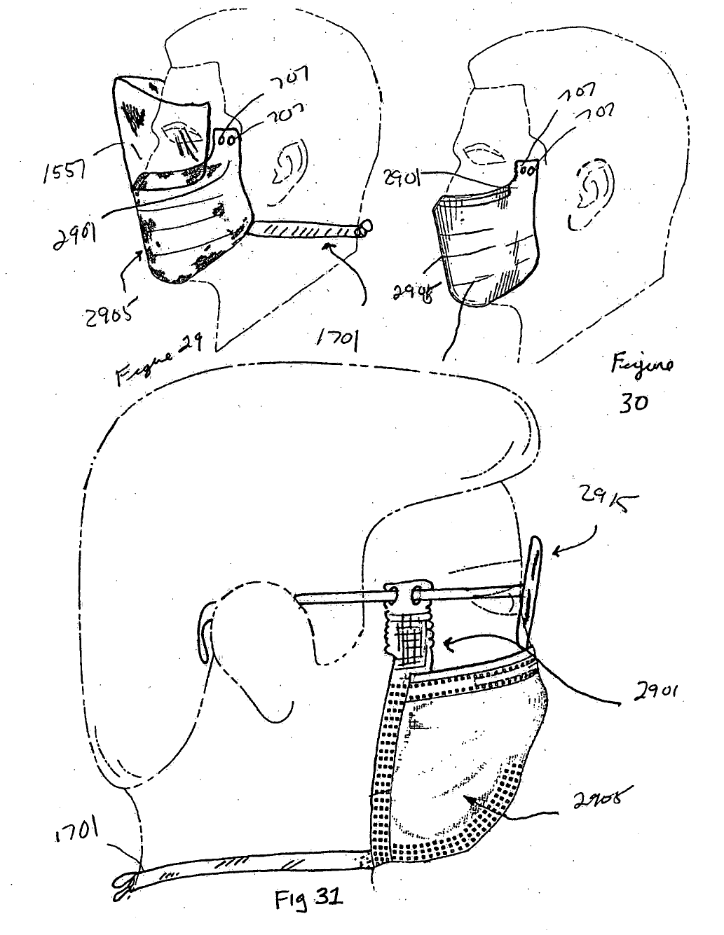

FIG. 32 illustrates a top view of another frame member with no projection members, and FIG. 33 illustrates a side view of the frame member.

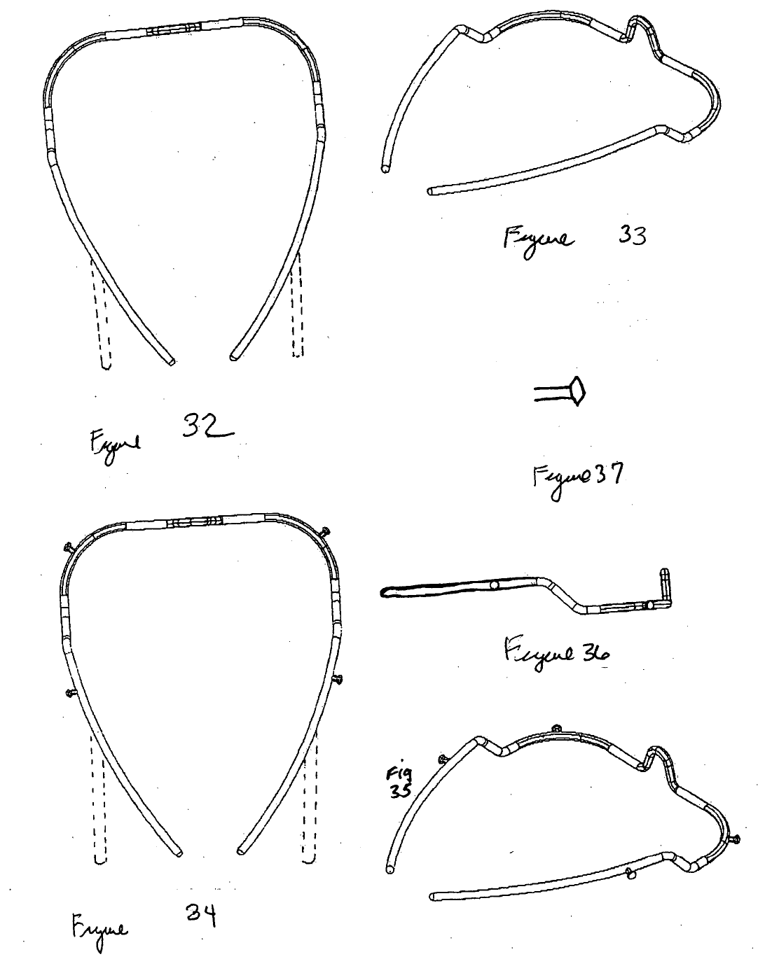

FIG. 34 illustrates a top view of another frame member with outward projecting members, and FIG. 35 illustrates a perspective view of frame member with outward projecting members.

FIG. 36 illustrates a side view of the frame member.

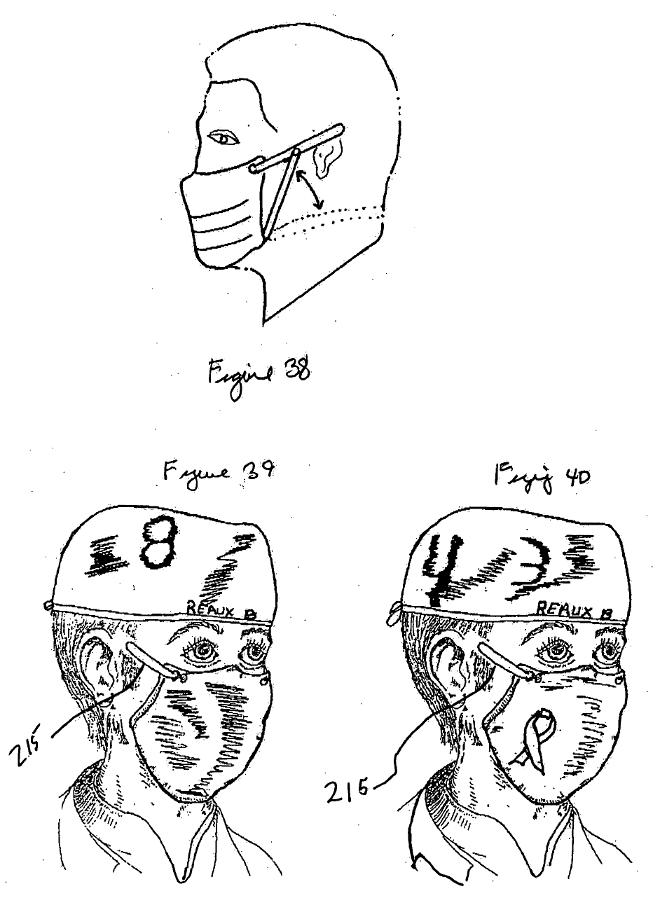

FIG. 37 illustrates a side view of the inward or outward projection member.

FIG. 38 illustrates a side view of another mask of the present invention where the lower cord member is connected to the frame member.

FIG. 39 illustrates a perspective view of a reversible mask where one side is blue.

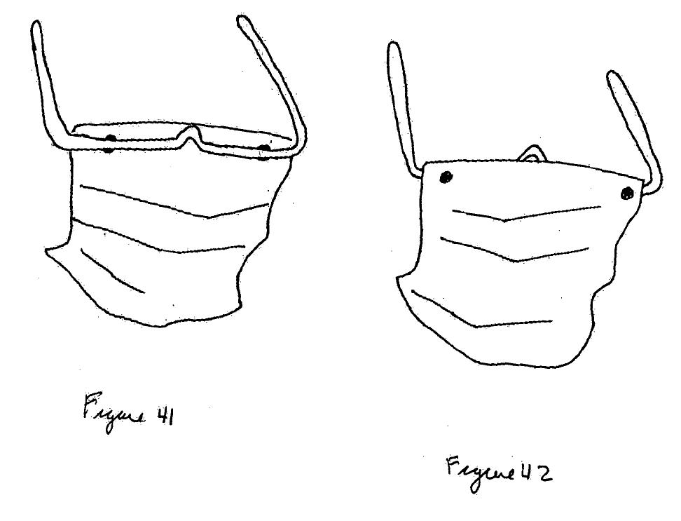

FIG. 40 illustrates a perspective view of a reversible mask where one side is pink with a breast-cancer logo.

FIG. 41 illustrates a frame member with the inwardly projection members extending inwards on the inner surface of the frame member.

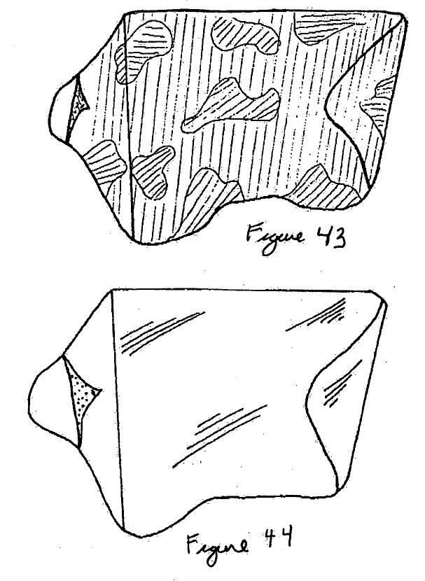

FIG. 42 illustrates a frame member with the outwardly projecting members extending outwards on the outer surface of the frame member.

FIG. 43 illustrates a protective cover with a camouflage design.

FIG. 4 illustrates a protective cover with a clear design.

The frame member may be rimless, and the frame member may be without a lens or may have a lens. The frame member may be attachable to more than one style of mask. The eye shield member may have an aperture to cooperate with the mask, or the eye shield member may have no apertures. The frame member may be single use or may be reusable. The frame member and mask and optional eye shield member may have use in the medical industry, industrial industry, army, hunting or other such applications. The lens cover may have a camouflage design to match the camouflage design on the mask. The frame design to be used in these different applications share many design criterion with alternative masks that will cooperate with the frame member. The mask may have a flat compact storage capability and may be quick to place on the frame member and remove. The lower head/neck tie may be placed on one side of the frame or be placed around the neck of the user. The mask may have a malleable nose-piece. The frame member may cooperate with the holes or may strap on to the top edge of the mask. The tie or elastic strap may hook into the side of the frame or coupled to the frame member. The lens cover may be clear or camouflage design and the cover may be removable and/or replaceable. The mask may be blue on one side and pink on the other side or may be orange on one side and camouflage on the other side. Other designs are within the scope of the invention. The frame member may have outward extending projections on the outside of the frame member or may have inwardly extending projections on the inside of the frame member. The frame member may be adapted to be wiped clean after use by any cleaning apparatus. The mask may be reversible and the mask and eye shield member may be disposable. The mask may be used with the eye shield member or maybe used without the eye shield member. The eye shield member may be anti-fog and may be antiglare. The mask and eye shield member may be convertible. The frame may not go completely around the head of the user. The frame may be one integral unit. The frame and mask may be a kit or it may be an integral unit. The frame may be formed of wood, plastic, paper, metal or the like or any combination. The unit, namely the eye shield member, the frame member or the mask, may be disposable in all or in part. The frame may have a nose pad and can have ear pads. The shield may be flexible and the frame may be flexible. The frame member may be enclosed but flexible. The frame member may be of color. The frame member may be multipurpose.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A mask and shield assembly for protecting the face of a user, comprising:
a reversible mask having an attachment device;
a frame member for detachably connecting to the attachment device of the reversible mask;
wherein the reversible mask includes an aperture;
wherein the outwardly extending projection cooperates with the aperture to hold the reversible mask in position.

2. A mask and shield assembly for protecting the face of a user as in claim 1, wherein the reversible mask includes a first surface of the reversible mask including camouflage indicia.

3. A mask and shield assembly for protecting the face of a user as in claim 2, wherein the reversible mask includes a second surface including hunter orange indicia.

4. A mask and shield assembly for protecting the face of a user as in claim 1, wherein the frame member includes a outwardly extending member on the outer surface of the frame member.

5. A mask and shield assembly for protecting the face of a user as in claim 1, wherein the frame member includes a inwardly extending member on the inner surface of the frame member.

6. A mask and shield assembly for protecting the face of a user as in claim 1, wherein the frame member has a substantially smooth surface.

7. A mask and shield assembly for protecting the face of a user as in claim 1, wherein the frame member includes a concave curved portion.

8. A mask and shield assembly for protecting the face of a user as in claim 1, wherein the frame member is flexible.

9. A mask and shield assembly for protecting the face of a user as in claim 1, wherein the attachment device includes an adhesive surface.

10. A mask and shield assembly for protecting the face of a user as in claim 9, wherein the attachment surface is protected by a protective sheet.

11. A mask and shield assembly for protecting the face of a user as in claim 9, wherein the adhesive surface is positioned on a front and back of the adhesive device.

12. A mask and shield assembly for protecting the face of a user as in claim 1, wherein the mask includes an expandable edge member.

13. A mask and shield assembly for protecting the face of a user as in claim 12, wherein the expandable edge member is positioned on a lower edge of the mask.

14. A mask and shield assembly for protecting the face of a user as in claim 12, wherein the expandable edge member is positioned along a side edge of the mask.

15. A mask and shield assembly for protecting the face of a user as in claim 1, wherein the reversible mask is affixed to the eye shielding member.

16. A mask and shield assembly for protecting the face of a user as in claim 1, wherein the shielding eye member includes a foldable side member.

17. A mask and shield assembly for protecting the face of a user as in claim 1, wherein the shielding eye member includes a protective cover.

18. A mask and shield assembly for protecting the face of a user as in claim 17, wherein the protective cover includes a tab member.

19. A mask and shield assembly for protecting the face of a user as in claim 1, wherein the reversible mask includes a lower cord member.

20. A mask and shield assembly as in claim 1, wherein the frame member includes a notch for the nose of the user.
